(19) United States
(12) Patent Application Publication
(10) Pub. No.: US 2023/0293763 A1
(43) Pub. Date: Sep. 21, 2023

(54) Antimicrobial Diapering Device

(71) Applicant: Sandie Pritchett, Glenview, IL (US)

(72) Inventor: Sandie Pritchett, Glenview, IL (US)

(21) Appl. No.: 17/697,424

(22) Filed: Mar. 17, 2022

(51) Int. Cl. A61L 15/44; A61F 13/84; A61L 15/40; A61L 15/20; A61F 13/56; A61K 35/644; A61K 31/122; A61K 31/121

(52) U.S. Cl. CPC A61L 15/44; A61F 13/5633; A61F 13/8405; A61K 31/121; A61K 31/122; A61K 35/644; A61L 15/20; A61L 15/40; A61L 2300/216; A61L 2300/30; A61L 2300/404

(57) ABSTRACT

An antimicrobial diapering device for one or more of inhibiting growth of a microorganism and killing a microorganism includes a diaper for diapering a user. The diaper comprises an outer layer, an inner layer, and a medial layer. The outer layer is substantially impervious to water. The inner layer is porous or wicking. The inner layer is impregnated with one or more of honey, an anthraquinone, and curcumin. The one or more of the honey, the anthraquinone, and the curcumin effect one or more of inhibiting growth of a microorganism and killing a microorganism. The medial layer is hydrophilic and is positioned between the outer layer and the inner layer. The medial layer absorbs moisture from urine and feces.

### (B) CROSS-REFERENCE TO RELATED APPLICATIONS

Not Applicable

### (C) STATEMENT REGARDING FEDERALLY SPONSORED RESEARCH OR DEVELOPMENT

Not Applicable

### (D) THE NAMES OF THE PARTIES TO A JOINT RESEARCH AGREEMENT

Not Applicable

### (E) INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### (F) STATEMENT REGARDING PRIOR DISCLOSURES BY THE INVENTOR OR JOINT INVENTOR

Not Applicable

### (G) BACKGROUND OF THE INVENTION

### Field of the Invention

The disclosure relates to diapering devices and more particularly pertains to a new diapering device for one or more of inhibiting growth of a microorganism and killing a microorganism. The present invention discloses a diapering device comprising a diaper comprising one or more of honey, an anthraquinone, and curcumin.

### Description of Related Art Including Information Disclosed Under 37 CFR 1.97 and 1.98

The prior art relates to diapering devices. Prior art diapering devices may comprise lotions and antimicrobials such as silver, hop extracts, and bacitracin. What is lacking in the prior art is a diapering device comprising a diaper comprising one or more of honey, an anthraquinone, and curcumin.

### (H) BRIEF SUMMARY OF THE INVENTION

An embodiment of the disclosure meets the needs presented above by generally comprising a diaper, which is configured for use in diapering a user. The diaper comprises an outer layer, an inner layer, and a medial layer. The outer layer is substantially impervious to water. The inner layer is porous or wicking. The inner layer is impregnated with one or more of honey, an anthraquinone, and curcumin. The one or more of the honey, the anthraquinone, and the curcumin are configured for one or more of inhibiting growth of a microorganism and killing a microorganism. The medial layer is hydrophilic and is positioned between the outer layer and the inner layer. The medial layer is configured to absorb moisture from urine and feces.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### I BRIEF DESCRIPTION OF SEVERAL VIEWS OF THE DRAWINGS

The disclosure will be better understood and objects other than those set forth above will become apparent when consideration is given to the following detailed description thereof. Such description makes reference to the annexed drawings wherein:

FIG. 1 is an isometric perspective view of an antimicrobial diapering device according to an embodiment of the disclosure.

FIG. 2 is a front view of an embodiment of the disclosure.

FIG. 3 is a rear view of an embodiment of the disclosure.

FIG. 4 is a side view of an embodiment of the disclosure.

FIG. 5 is a cross-sectional view of an embodiment of the disclosure.

FIG. 6 is an in-use view of an embodiment of the disclosure.

FIG. 7 is a flow diagram for a method utilizing an embodiment of the disclosure.

### (J) DETAILED DESCRIPTION OF THE INVENTION

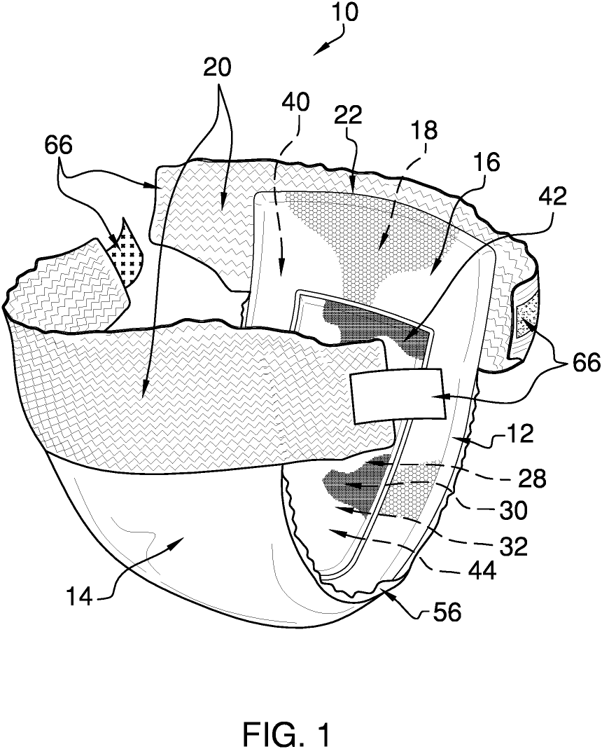

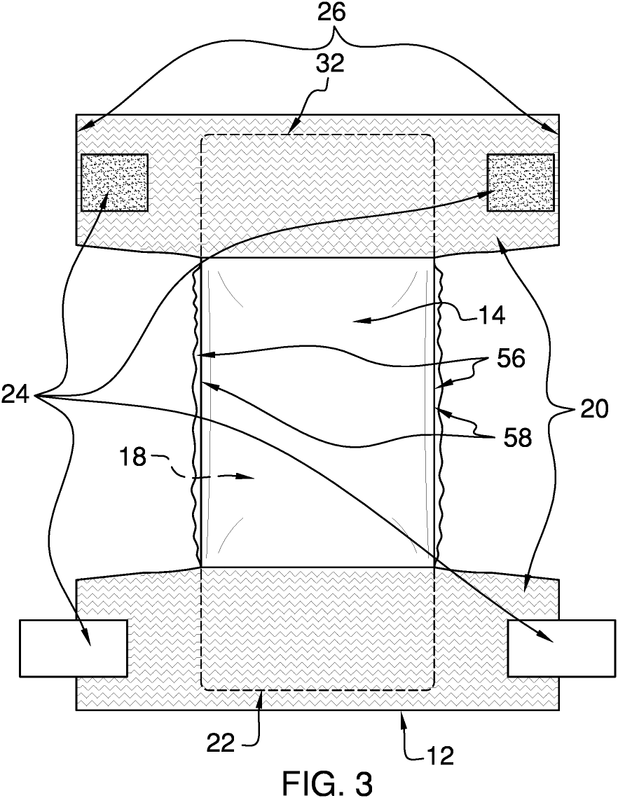

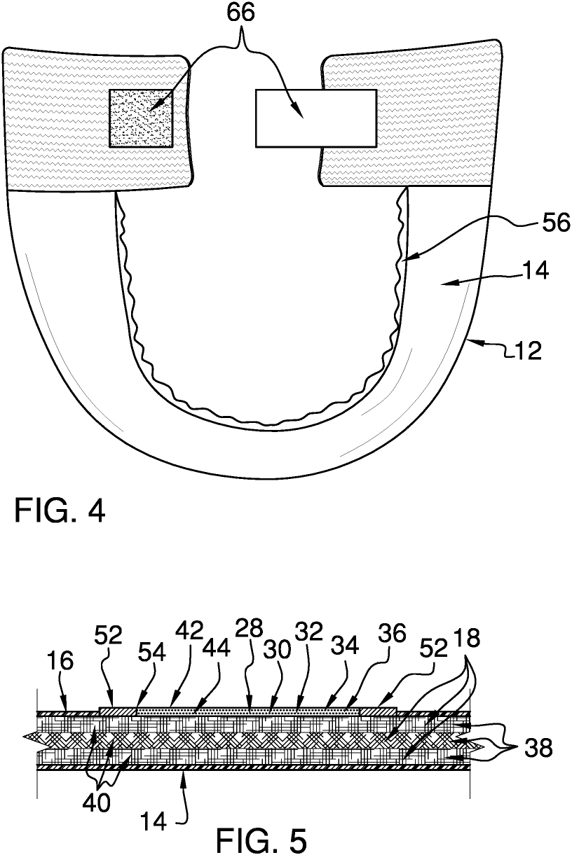

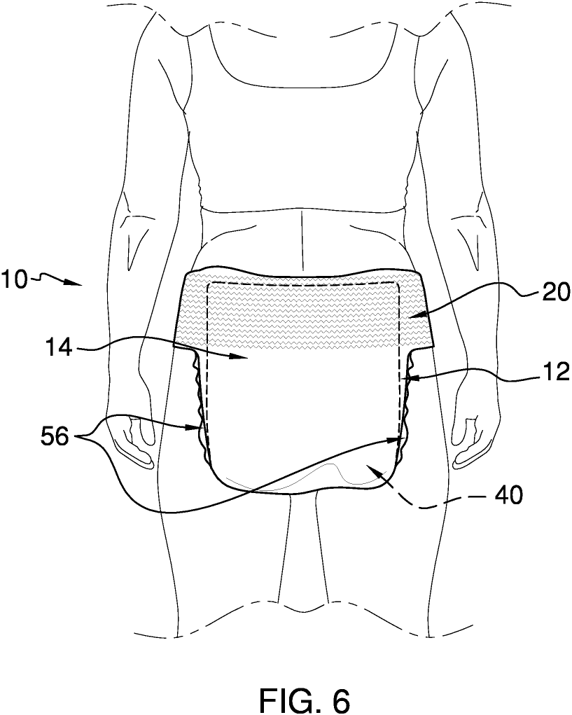

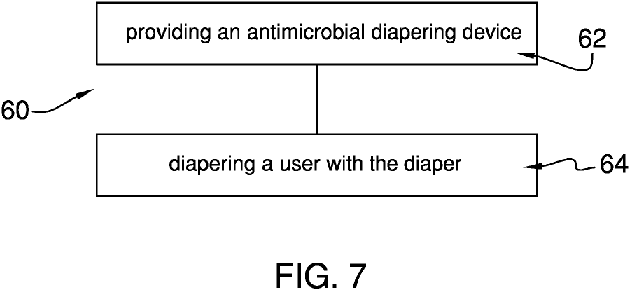

With reference now to the drawings, and in particular to FIGS. 1 through 7 thereof, a new diapering device embodying the principles and concepts of an embodiment of the disclosure and generally designated by the reference numeral 10 will be described.

As best illustrated in FIGS. 1 through 7, the antimicrobial diapering device 10 generally comprises a diaper 12, which is configured for use in diapering a user. The diaper 12 comprises an outer layer 14, an inner layer 16, and a medial layer 18. The outer layer 14 is substantially impervious to water. Each of a pair of bands 20 is engaged to a respective opposed end 22 of the outer layer 14 and extends bidirectionally from the respective opposed end 22. The bands 20 comprise elastomer, silicone, or rubber such that the bands 20 are resiliently stretchable.

Each of a plurality of fasteners 24 is engaged to an opposing end 26 of a respective band 20 so that a fastener 24 is engaged each opposing end 26 of each band 20. The fasteners 24 are selectively mutually couplable so that a respective fastener 24 engaged to one of the bands 20 is positioned to selectively engage an associated fastener 24 positioned on the other of the bands 20. The fasteners 24 thus are configured to secure the diaper 12 to the user. The respective fastener 24 and the associated fastener 24 may comprise a hook and loop fastener 66, or other fastening means, such as, but not limited to, adhesive, clips, and the like.

The inner layer 16 is porous or wicking. The inner layer 16 is impregnated with one or more of honey 28, an anthraquinone 30, and curcumin 32. The one or more of the honey 28, the anthraquinone 30, and the curcumin 32 are configured for one or more of inhibiting growth of a microorganism and killing a microorganism. The honey 28 is in powder form. The anthraquinone 30 is present as aloe vera powder 34. The curcumin 32 is present as turmeric powder 36.

The medial layer 18 is hydrophilic and is positioned between the outer layer 14 and the inner layer 16. The medial layer 18 is configured to absorb moisture from urine and feces. The medial layer 18 may comprise three strata 38 of absorbent material 40, or other number of strata 38 of absorbent material 40, such as, but not limited to, two strata 38 or four strata 38. A variety of absorbent materials 40 are anticipated by the present invention, such as, but not limited to, cotton, hemp, wool, sodium polyacrylate, and the like.

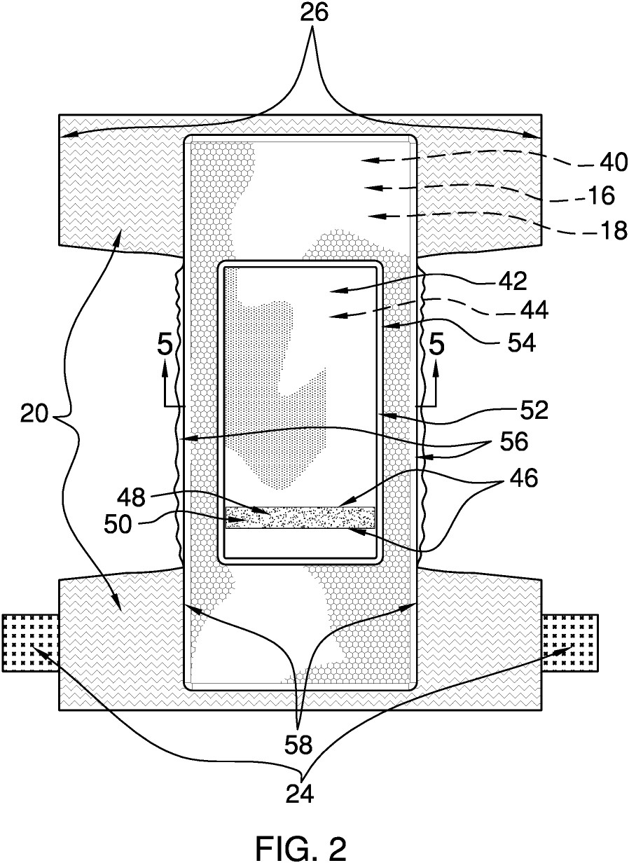

As shown in FIG. 2, a panel 42 is engaged to the inner layer 16 so that the panel 42 and the inner layer 16 define a cavity 44. The panel 42, like the inner layer 16, is porous or wicking. The one or more of the honey 28, the anthraquinone 30, and the curcumin 32 is positioned in the cavity 44.

A pair of subpanels 46 is engaged to and extends between the inner layer 16 and the panel 42 to defines a compartment 48 within the cavity 44. Powdered black soap 50 is positioned in the compartment 48. The powdered black soap 50 also is configured for one or more of inhibiting growth of a microorganism and killing a microorganism.

Shea butter 52 is positioned on one or more of the inner layer 16 and the panel 42, as shown in FIG. 2. The shea butter 52 extends substantially around a circumference 54 of the panel 42. The shea butter 52 is configured to moisturize skin of the user and for one or more of inhibiting growth of a microorganism and killing a microorganism.

Each of a pair of flaps 56 is engaged to a respective opposed edge 58 of the outer panel 42 and extends between the bands 20. The flaps 56 comprises elastomer, silicone, or rubber so that the flaps 56 are resiliently stretchable. The flaps 56 are configured to sealably engage thighs of the user upon donning of the diaper 12, thereby inhibiting leakage.

In use, the antimicrobial diapering device 10 enables a method 60 of preventing or treating a skin infection in a diapered user. The method 60 comprises a first step 62 of providing an antimicrobial diapering device 10 according to the specification above. A second step 64 of the method 60 is diapering the user with the diaper 12.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An antimicrobial diapering device comprising a diaper configured for use in diapering a user, the diaper comprising:
an outer layer substantially impervious to water;
an inner layer impregnated with one or more of honey, an anthraquinone, and curcumin, the inner layer being porous or wicking, wherein the one or more of honey, an anthraquinone, and curcumin are configured for one or more of inhibiting growth of a microorganism and killing a microorganism; and
a medial layer positioned between the outer layer and the inner layer, the medial layer being hydrophilic, wherein the medial layer is configured for absorbing moisture from urine and feces.

2. The antimicrobial diapering device of claim 1, further including:
a pair of bands, each band being engaged to a respective opposed end of the outer layer and extending bidirectionally from the respective opposed end; and
a plurality of fasteners, each fastener being engaged to an opposing end of a respective band, such that a fastener is engaged each opposing end of each band, the fasteners being selectively mutually couplable, such that a respective fastener engaged to one of the bands is positioned for selectively engaging an associated fastener positioned on the other of the bands, wherein the fasteners are configured for securing the diaper to the user.

3. The antimicrobial diapering device of claim 2, further wherein the bands comprise elastomer, silicone, or rubber such that the bands are resiliently stretchable.

4. The antimicrobial diapering device of claim 2, further wherein the respective fastener and the associated fastener comprise a hook and loop fastener.

5. The antimicrobial diapering device of claim 1, further wherein:
the honey being in powder form;
the anthraquinone being present as aloe vera powder; and
the curcumin being present as turmeric powder.

6. The antimicrobial diapering device of claim 1, further wherein the medial layer comprises three strata of absorbent material.

7. The antimicrobial diapering device of claim 5, further including:
a panel engaged to the inner layer, such that the panel and the inner layer define a cavity, the panel being porous or wicking; and
the one or more of the honey, the anthraquinone, and the curcumin being positioned in the cavity.

8. The antimicrobial diapering device of claim 7, further including:
a pair of subpanels engaged to and extending between the inner layer and the panel defining a compartment within the cavity; and
powdered black soap being positioned in the compartment, wherein the powdered black soap is configured for one or more of inhibiting growth of a microorganism and killing a microorganism.

9. The antimicrobial diapering device of claim 7, further including shea butter positioned on one or more of the inner layer and the panel, the shea butter extending substantially around a circumference of the panel, wherein the shea butter is configured for moisturizing skin of the user and for one or more of inhibiting growth of a microorganism and killing a microorganism.

10. The antimicrobial diapering device of claim 2, further including a pair of flaps, each flap being engaged to a respective opposed edge of the outer panel and extending between the bands, the flaps comprising elastomer, silicone, or rubber such that the flaps are resiliently stretchable, wherein the flaps are configured for sealably engaging thighs of the user upon donning of the diaper.

11. A method of preventing or treating a skin infection in a diapered user, the method comprising the steps of:
providing an antimicrobial diapering device comprising a diaper configured for use in diapering a user, the diaper comprising:
an outer layer substantially impervious to water,
an inner layer impregnated with one or more of honey, an anthraquinone, and curcumin, the inner layer being porous or wicking, wherein the one or more of the honey, the anthraquinone, and the curcumin are configured for one or more of inhibiting growth of a microorganism and killing a microorganism, and
a medial layer positioned between the outer layer and the inner layer, the medial layer being hydrophilic, wherein the medial layer is configured for absorbing moisture from urine and feces; and
diapering the user with the diaper.

12. An antimicrobial diapering device comprising:
a diaper configured for use in diapering a user, the diaper comprising:
an outer layer substantially impervious to water,
a pair of bands, each band being engaged to a respective opposed end of the outer layer and extending bidirectionally from the respective opposed end, the bands comprising elastomer, silicone, or rubber such that the bands are resiliently stretchable,
a plurality of fasteners, each fastener being engaged to an opposing end of a respective band, such that a fastener is engaged each opposing end of each band, the fasteners being selectively mutually couplable, such that a respective fastener engaged to one of the bands is positioned for selectively engaging an associated fastener positioned on the other of the bands, wherein the fasteners are configured for securing the diaper to the user, the respective fastener and the associated fastener comprising a hook and loop fastener,
an inner layer impregnated with one or more of honey, an anthraquinone, and curcumin, the inner layer being porous or wicking, wherein the one or more of the honey, the anthraquinone, and the curcumin are configured for one or more of inhibiting growth of a microorganism and killing a microorganism, the honey being in powder form, the anthraquinone being present as aloe vera powder, the curcumin being present as turmeric powder, and
a medial layer positioned between the outer layer and the inner layer, the medial layer being hydrophilic, wherein the medial layer is configured for absorbing moisture from urine and feces, the medial layer comprising three strata of absorbent material;
a panel engaged to the inner layer, such that the panel and the inner layer define a cavity, the one or more of the honey, the anthraquinone, and the curcumin being positioned in the cavity, the panel being porous or wicking;
a pair of subpanels engaged to and extending between the inner layer and the panel defining a compartment within the cavity;
powdered black soap being positioned in the compartment, wherein the powdered black soap is configured for one or more of inhibiting growth of a microorganism and killing a microorganism;
shea butter positioned on one or more of the inner layer and the panel, the shea butter extending substantially around a circumference of the panel, wherein the shea butter is configured for moisturizing skin of the user and for one or more of inhibiting growth of a microorganism and killing a microorganism; and
a pair of flaps, each flap being engaged to a respective opposed edge of the outer panel and extending between the bands, the flaps comprising elastomer, silicone, or rubber such that the flaps are resiliently stretchable, wherein the flaps are configured for sealably engaging thighs of the user upon donning of the diaper.
